SEQUENCE LISTING (19 sequences)

SEQ ID NO: 1 - RNA, 50 nt, Renilla reniformis
uaggcuagccaccauggcuuccaagguguacgaccccgagcaacgcaaac

SEQ ID NO: 2 - RNA, 30 nt, Renilla reniformis
ugguaccgaagguuccacaugcuggggcuc

SEQ ID NO: 3 - RNA, 30 nt, Renilla reniformis
cucggggucguacaccuuggaagccauggu

SEQ ID NO: 4 - DNA, 44 bp, Renilla reniformis
ctcgagtggtaccgaaggttccacatgctggggctcgcggccgc

SEQ ID NO: 5 - DNA, 44 bp, Renilla reniformis
ctcgagctcggggtcgtacaccttggaagccatggtgcggccgc

SEQ ID NO: 6 - DNA, 13 bp, Artificial Sequence
  Description of Artificial Sequence Synthetic oligonucleotide
ctgataatgtatc

SEQ ID NO: 7 - DNA, 13 bp, Artificial Sequence
  Description of Artificial Sequence Synthetic oligonucleotide
gatacattatcag

SEQ ID NO: 8 - DNA, 13 bp, Artificial Sequence
  Description of Artificial Sequence Synthetic oligonucleotide
ctatgtaatagtc

SEQ ID NO: 9 - DNA, 13 bp, Artificial Sequence
  Description of Artificial Sequence Synthetic oligonucleotide
gactattacatag

SEQ ID NO: 10 - RNA, 13 nt, Artificial Sequence
  Description of Artificial Sequence Synthetic oligonucleotide
cugauaauguauc

SEQ ID NO: 11 - RNA, 13 nt, Artificial Sequence
  Description of Artificial Sequence Synthetic oligonucleotide
gacuauuacauag

SEQ ID NO: 12 - DNA, 32 bp, Artificial Sequence
  Description of Artificial Sequence Synthetic oligonucleotide
cccctcgagctcggggtcgtacaccttggaag

SEQ ID NO: 13 - DNA, 27 bp, Artificial Sequence
  Description of Artificial Sequence Synthetic oligonucleotide
cccgcggccgcaccatggcttccaagg

SEQ ID NO: 14 - DNA, 30 bp, Artificial Sequence
  Description of Artificial Sequence Synthetic oligonucleotide
cccctcgagtggtaccgaaggttccacatg

SEQ ID NO: 15 - DNA, 31 bp, Artificial Sequence
  Description of Artificial Sequence Synthetic oligonucleotide
cccgcggccgcgagccccagcatgtggaacc

SEQ ID NO: 16 - DNA, 19 bp, Artificial Sequence
  Description of Artificial Sequence Synthetic primer
gagaaaattagtagatttc

SEQ ID NO: 17 - DNA, 160 bp, Drosophila melanogaster
aaaattaggagcacaagaagaatttgttgatgtccatatcaatgcttcaagacgcacaaacgcgaaccttagctgctgcattagctggcataaaacaagaggacgttcatctagaccgttccatgtcgctatcgccccccatgtcggccaacacatcagc

SEQ ID NO: 18 - DNA, 160 bp, Artificial Sequence
  Description of Artificial Sequence Synthetic nucleotide construct
ttttaatcctcgtgttcttcttaaacaactacaggtatagttacgaagttctgcgtgtttgcgcttggaatcgacgacgtaatcgaccgtattttgttctcctgcaagtagatctggcaaggtacagcgatagcggggggtacagccggttgtgtagtcg

SEQ ID NO: 19 - RNA, 30 nt, Renilla reniformis
accauggcuuccaagguguacgaccccgag

(19) United States
(12) Patent Application Publication
(10) Pub. No.: US 2007/0092499 A1
(43) Pub. Date: Apr. 26, 2007

(54) Method for changing genetic properties of eukaryotic organism

(75) Inventor: Nikolai Andreevich Churikov, Moscow (RU)

Correspondence Address: BUCHANAN, INGERSOLL & ROONEY PC, POST OFFICE BOX 1404, ALEXANDRIA, VA, 22313-1404, US

(73) Assignee: INSTITUT MOLEKULYARNOI BIOLOGII IM. V.A. ENGELGARDTA ROSSIISKOI AKADEMII NAUK, Moscow (RU)

(21) Appl. No.: 11/500,910

(22) Filed: Aug. 9, 2006

Related U.S. Application Data
(63) Continuation-in-part of application No. 09/913,040, filed on Aug. 9, 2001, now abandoned.

(30) Foreign Application Priority Data
Dec. 9, 1999 (RU) 99125827

(51) Int. Cl. A61K 48/00; C12N 15/09

(52) U.S. Cl. 424/93.21; 514/44; 435/455

(57) ABSTRACT

The invention provides a method for changing the genetic properties of an eukaryotic organism by suppression of expression of a target gene. Proposed method can be used in gene-therapy in medicine, agriculture, or industrial biotechnology. Genetic properties of an eukaryotic organism are changed by gene-specific silencing of a selected gene using small RNA molecules, that are complementary in a parallel orientation (pcRNAs) to mRNA of the selected gene; wherein pcRNAs are synthesized in vivo or in vitro on the artificial DNA sequence encoding pcRNA, said artificial DNA sequence possesses symmetrical nucleotide ordering (mirror inversion) in respect to the nucleotide sequence of the gene.

### FIELD OF THE INVENTION

The invention relates to molecular biology, molecular genetics and biotechnology, and can be used for the gene-therapy in the medical agricultural or the biotechnology industries. The invention relates to gene-specific silencing of the disease-related genes or the genes interfering a buildup of a product, respectively.

### BACKGROUND

Various methods for changing the genetic properties of an organism are known. Some methods cause the damage of a gene. An example is the “gene knockout”. This method uses the damage mutation of a selected gene in germ-line or stem cells, and thus cannot be used in most cases for a developed organism [L. V. Varga, S. Toth, I. Novak, A. Falus, Immunol. Lett., 1999, vol. 69, p. 217; J. Osada, N. Maeda, Methods Mol. Biol., 1998, vol. 110, p. 79].

In recent years, increased attention has been given to another method for changing the genetic properties of an organism by RNA interference, leading to gene-specific silencing by changes to the regulation of an undamaged gene [M. K. Montgomery, A. Fire, Trends in Genetics, 1998, vol. 14, p. 255; P. Sharp, Genes & Development, 1999, vol. 13, p. 139]. RNA interference can be used for gene-specific silencing at any stage of development, including in fully developed adults. The increased attention to RNA interference is due to the fact that these studies serendipitously uncovered the ancient mechanisms of gene regulation. The physiological role of this mechanism of regulation may include the local changes of chromosomal structure, transcription activity, RNA processing, transport into the cytoplasm, and RNA stability.

Until now, RNA interference leading to gene-specific silencing was described in different organisms, including nematode, Drosophila, fungi, and plants.

The known method for changes of genetic properties of an organism based on RNA interference uses the antisense RNA (asRNA) that is complementary to the mRNA of the selected gene in antiparallel orientation, and is synthesized in vitro and introduced into the organism [A. Fire, S- Q. Xu, M. K. Montgomery, S. V. Kostas, S. E. Driver, C. C. Mello, Nature, 1998, vol. 391, p. 806].

This described method is carried out as follows:
1. A gene with pathogenic activity is selected;
2. A DNA construct possessing a selected gene or its cDNA (a sequence corresponding to mRNA), i.e. natural DNA, in the opposite polarity under the control of selected promoter is prepared. This permits to perform transcription of non-coding strand of the gene. For the generation of the construct different vectors are used possessing DNA sequences for selection of transformants, for efficient expression of the turned-over gene and for integration of the construct in chromosomal domains and the proper expression of the DNA construct;
3. asRNA is synthesized in vitro on the construct and introduced into organism by different methods (electroporation, injections, per os).

One concern with this method for changing genetic properties of an organism by RNA interference, is the common occurrence of reversions of the constructs designed for asRNA synthesis by rearrangements, which leads to the stopping of asRNA transcription and the start of transcription of the sequences corresponding to the mRNA-strand. Thus, instead of inhibition of the activity of the selected gene, an increased transcription of the gene often occurs. The start of transcription of the sequences corresponding to mRNA-strand can also happen if the host promoter sequences transcribing the sense strands are present in the target site of insertion of the construct. The probability of such events is high.

The common occurrence of reversions is illustrated by demonstrative experiments on transgenic organisms. The constructs in these cases were introduced with the opposite aim, i.e., to increase the activity of a selected gene. However, reversions by spontaneous activation of transcription from the opposite strand resulted in complete inhibition of gene activity instead of activation of its expression, i.e. to gene-specific silencing by RNA interference mechanisms [M. K. Montgomery, A. Fire, Trends in Genetics, 1998, vol. 14, p. 255; P. Sharp, Genes & Development, 1999, vol. 13, p. 139].

### SUMMARY OF THE INVENTION

The present invention provides methods for changing the genetic properties of an organism by gene-specific silencing of a selected gene. Small RNA molecules that are complementary in a parallel orientation (pcRNA) to mRNA of the selected gene are used. pcRNA are synthesized in vivo or in vitro on the artificial DNA sequence encoding pcRNA. The artificial DNA sequence possesses symmetrical nucleotide ordering (mirror inversion) in respect to the nucleotide sequence of the gene. The relationship between genes encoding target sequences, asRNA and pcRNA, as well as between mRNA, asRNA and pcRNA, are illustrated by the following figure:

| | | |
|---|---|---|
| GENE, | | |
| encoding target | GENE, | GENE, |
| sequence | encoding asRNA | encoding pcRNA |

| | | | | | |
|---|---|---|---|---|---|
| 5′ AGTC 3′ | (+) | 5′ GATC 3′ | (+) | 5′ TCAG 3′ | (+) |
| 3′ TCAG 5′ | (−) | 3′ CTAG 3′ | (−) | 3′ AGTC 3′ | (−) |

| mRNA | asRNA | pcRNA |
|---|---|---|
| 5′ AGTC 3′ | 5′ GATC 3′ | 5′ TCAG 3′ |

wherein:
asRNA—antisence RNA;
pcRNA—RNA, that are complementary in a parallel orientation to mRNA;
(+)—“+” strand of the gene;
(−)—“−” strand of the gene.

It is believed that pcRNA and mRNA form parallel RNA-RNA duplexes in vivo. The physical capacity of RNA molecules to form parallel RNA-RNA duplexes was demonstrated in in vitro experiments [N. A. Tchurikov, N. A. Ponomarenko, Y. B. Golova, B. K. Chernov, J. Biomol. Struct. & Dynamics, 1995, vol. 13, p. 507; N. A. Tchurikov, L. G. Chistyakova, G. B. Zavilgelsky, I. V. Manukhov, J. Biol. Chem., 2000, vol. 275, p. 26523]. It was demonstrated that the regions of both molecules are protected from a strong treatment with SI endonuclease only after annealing of mRNA to the corresponding pcRNA. The properties of parallel-stranded DNA were also described [N. A. Tchurikov, Genetica, 1992, vol. 87, p. 113; N. A. Tchurikov, A. K. Schyolkina, O. F. Borissova, B. K. Chernov, FEBS Letters, vol. 297, p. 233; O. F. Borissova, A. K. Schyolkina, B. K. Chernov, N. A. Tchurikov, FEBS Letters, vol. 322, p. 304]. It is likely that the extremely sensitive system responsible for monitoring of RNA molecules in the cell recognizes the parallel RNA-RNA duplexes and triggers the specific degradation of mRNA involved in the generation of the duplexes, or leads to translational silencing, reminding the RNAi mechanisms involving short molecules of siRNAs or miRNAs [N. A. Tchurikov, Biochemistry (Mosc), 2005, vol. 70, p. 406].

The present invention provides a method for changing the genetic properties of an eukaryotic organism by altering or preventing expression of a target gene, comprising (a) selecting a target gene, and (b) introducing into the organism pcRNA molecules that are complementary in a parallel orientation to mRNA of the target gene or a fragment thereof. The pcRNA molecules complementary in a parallel orientation to mRNA of the target gene or fragment thereof may be synthesized in vitro prior to introduction into the organism, and may be synthesized using an artificial DNA sequence as a template. The pcRNA molecules may be introduced into the organism via injection. The pcRNA molecules may also be introduced into the organism via introduction into the organism of a DNA construct that expresses the pcRNA molecules that are complementary in a parallel orientation to mRNA of the target gene or fragment thereof in vivo.

The DNA construct may comprise a suitable promoter and a DNA sequence encoding pcRNA, wherein said DNA sequence possesses symmetrical nucleotide ordering with respect to the nucleotide sequence of the target gene; and wherein said DNA sequence is under control of the promoter. The DNA construct may further comprise sequences that enable integration of the DNA construct into the chromosome of the organism as well as the proper expression of the DNA construct.

The pcRNA molecules complementary in a parallel orientation to mRNA of the target gene or fragment thereof may be introduced into the organism via ex vivo gene therapy. The ex vivo gene therapy may comprise (a) removing cells from the organism, (b) transforming the cells with a vector comprising a DNA construct that expresses the pcRNA molecules that are complementary in a parallel orientation to mRNA of the target gene or fragment thereof in vivo, to produce genetically modified cells, and (c) injecting the genetically modified cells into the organism. The DNA construct may comprise a suitable promoter and a DNA sequence encoding pcRNA, wherein the DNA sequence encoding pcRNA is under control of a promoter. The DNA construct may further comprise sequences that enable integration of the DNA construct into the chromosome of the organism and the proper expression of the DNA construct.

The pcRNA molecules that are complementary in a parallel orientation to mRNA of the target gene or a fragment thereof may interact with the mRNA of the target gene, thereby altering or preventing expression of the target gene. Preferably, the target gene has undesirable activity.

### BRIEF DESCRIPTION OF THE DRAWINGS

FIG. 1 shows the structure of the DNA construct possessing a mirror nucleotide sequence with respect to the Drosophila Kruppel gene and designed for expression of pcRNA (Kr-par), where:

a—the relationship between the nucleotide sequence of the Kruppel gene and the artificial chemically synthesized DNA, possessing mirror nucleotide sequence in respect the gene (+ strands correspond to Kr-par pcRNA or mRNA); the synthesized DNA in the construct is under the control of the T7 RNA polymerase promoter driving the expression of Kr-par pcRNA (synthesis on the opposite strand of the construct is driven by SP6 RNA polymerase promoter);

b—relationship between Kruppel mRNA and Kr-par pcRNA.

FIG. 2 shows the phenotypes of normal larva and Kr phenocopies generated after injections of the Kr-par pcRNA, where:

a—phenotypes of normal Drosophila larva;

b—phenotype of the larva developed after injection of the Kr-par pcRNA and possessing deletions of adjacent thoracic and the first abdominal segments;

c—phenotype of the larva developed after injection of the Kr-par pcRNA and possessing deletions of adjacent thoracic and three anterior abdominal segments. The arrow shows the actopic tracheal ending that is characteristic only for the Kr phenotype.

FIG. 3 shows the design of DNA constructs possessing complementary stretches corresponding to the same 30 bp region of Renilla gene, where:

a—relationship between 30 nt fragment of Renilla luciferase mRNA coding region (SEQ ID NO: 1) and sequences of pcRNA (SEQ ID NO: 2) and asRNA (SEQ ID NO: 3), possessing complementary bases in parallel and antiparallel orientation, respectively;

b—schematic presentation of insertion into XhoI-NotI sites of psiCHECK-2 vector specifying pcRNA stretch (“par”); the filled bar shows the complementary region in Renilla luciferase gene (SEQ ID NO: 4);

c—schematic presentation of insertion into XhoI-NotI sites of psiCHECK-2 vector specifying asRNA stretch (“as”); the filled bar shows the complementary region in Renilla luciferase gene (SEQ ID NO: 5);

d—intramolecular structures that may be formed between the same coding region of Renilla luciferase mRNA and the complementary regions at its 3′ end corresponding to par or as stretches (numberings according the AY535007).

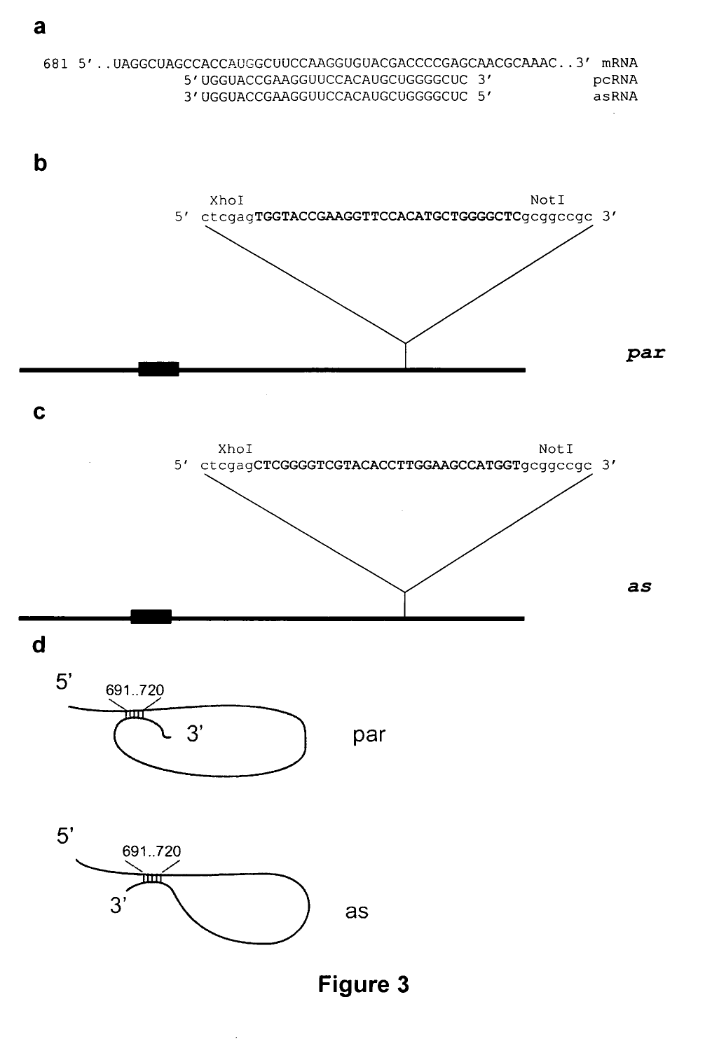

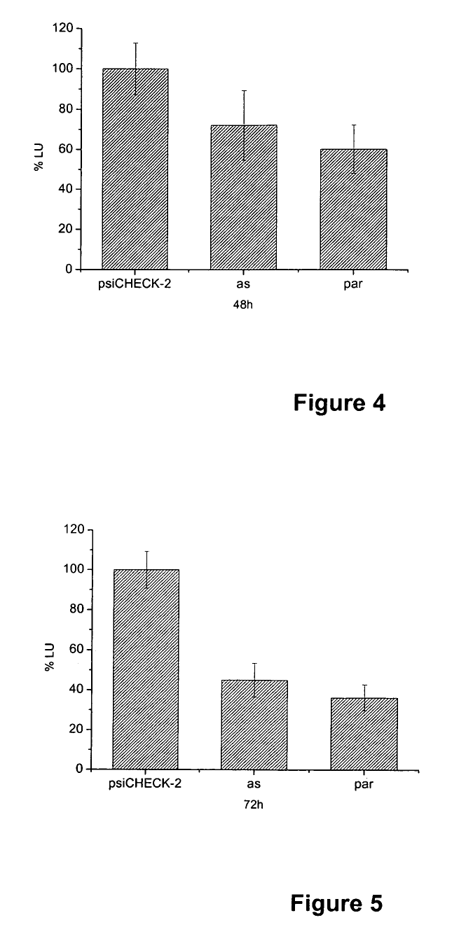

FIG. 4 shows the inhibition of Renilla gene expression 48 h after transfection with DNAs: vector without any insert (psiCHECK-2) and the constructs possessing XhoI-NotI inserts (FIGS. 3b, c) in the vector specifying Renilla luciferase mRNA possessing as or par stretches, respectively.

FIG. 5—The same as in FIG. 4, but after 72 hours after transfection.

### DETAILED DESCRIPTION

The present invention provides a way to increase the RNA interference reliability, while excluding the possibility of reversions leading to the synthesis of mRNA sequences on a construct designed for gene-specific silencing.

The present invention provides a method for changing genetic properties of eukaryotic organism and is based on the biological properties of mirror inversions of nucleotide sequences that are realized in gene-specific silencing. The claimed method is based on the potent and specific biological activity of the transcripts (pcRNA) coming from mirror inversions of nucleotide sequences in DNA. The present methods allow for changes in phenotype, and can be used, for example, for gene-specific silencing of disease-related genes, or genes causing a buildup of a product.

Adult cells may be used for ex vivo gene therapy using the constructs expressing pcRNA. The method allow for the patient's own cells to be removed and grown in the laboratory. These cells are exposed to the vector carrying the desired genetic construct expressing pcRNA. The vector enters the cells, and pcRNA may be expressed. It is possible to screen the cells for pcRNA expression and gene-specific silencing level before actually delivering them to patients in need. The genetically modified cells are grown in the laboratory, and then may be returned to the patient by appropriate method such as by injection into a vein. This method is called ex vivo, as the cells are grown outside the body. In these experiments, the patient's own cells are manipulated by gene therapy leading to insertion of genetic construct expressing pcRNA. In using this ex vivo method, two components are critical for the delivery of genetic constructs (i) the vector system and (ii) the cell type for grafting. Using an ex vivo method, it was possible to deliver CFTR gene for cystic fibrosis therapy and to establish the treatment of Parkinson's disease and rheumatoid arthritis [Wang G., Bunnell B. A., Painter R. G., Quiniones B. C., Tom. S., Lanson N. A., Jr., Spees J. L., Bertucci D., Peister A., Weiss D. J., Valentine V. G., Prockop D. J., Kolls J. K., Proc. Natl. Acad. Sci. U.S.A., 2005, vol. 102, p. 186; Ishida A., Yasuzumi F. Brain Dev., 2000, vol. 22, Suppl 1:S143-7; S. Takahashi, K. Ohsugi, T. Yamamoto, M. Shiomi, N. Sakuragawa, Tohoku, J. Exp. Med., 2001, vol. 193, p. 279].

The problem of delivery of nucleic acids into cells is a large problem for gene therapy. This problem is currently addressed by ex vivo procedures and stem cells technologies. However, the data on the rapid degradation of RNA molecules in the bloodstream are still controversial. Initially, a method for efficient in vivo intravenous delivery of siRNAs to organs of postnatal mice was described [D. L. Lewis, J. E. Hagstrom, A. G. Loomis, J. A. Wolf, H. Herweijer, 2002, Nature Genet,. vol. 32, p. 107]. Effective and specific inhibition of transgene expression in a variety of organs was observed. More recently a simple and effective method for the intravenous delivery of nucleic acids was described [Soutschek J, Akinc A, Bramlage B, Charisse K, Constien R, Donoghue M, Elbashir S, Geick A, Hadwiger P, Harborth J, John M, Kesavan V, Lavine G, Pandey R K, Racie T, Rajeev K G, Rohl I, Toudjarska I, Wang G, Wuschko S, Bumcrot D, Koteliansky V, Limmer S, Manoharan M, Vornlocher H P., Nature, 2004, vol. 432, p. 173]. It was concluded that the cholesterol-conjugate approach for therapeutic silencing of an endogenous gene could be achieved by systemic administration of modified RNAs [J. J. Rossi Nature, 2004, vol. 432, p. 155].

The claimed method is useful for changing genetic properties of an organism by gene-specific silencing of a selected gene using RNA molecules that are complementary in a parallel orientation (pcRNA) to mRNA of the selected gene; pcRNA are synthesized in vivo or in vitro on the artificial DNA sequence encoding pcRNA, wherein said artificial DNA sequence possesses symmetrical nucleotide ordering (mirror inversion) in respect to the nucleotide sequence of the gene.

The present method leads to more efficient gene-specific silencing and excludes the synthesis of mRNA sequences because in the constructs the non-homologous artificial DNA sequence is used.

The method may comprise the following steps:
1. A gene with pathogenic activity (leading to a disease or interfering a buildup of biotechnological product) is selected;
2. Artificial DNA sequence is chemically synthesized, where the said artificial DNA sequence encodes pcRNA with respect to the fragment of a selected gene;
3. A DNA construct is prepared on the basis of different vectors, possessing the chemically synthesized DNA sequence under the control of appropriate promoter, and a number of sequences important for integration of the DNA construct in chromosomal domains and for efficient expression of the insert;
4. The construct is introduced into an organism by different methods for in vivo synthesis of pcRNA (in vivo or ex vivo transformations), or pcRNA is synthesized in vitro and is used for injections.

The present mRNA and pcRNA may have complementary nucleotides in the same polarity, i.e., they are parallel 5′ to 3′. mRNA and antisense RNA have complementary bases as well, but the 5′-3′ text of mRNA corresponds the complementary text of antisense RNA in 3′-5′ polarity. The mRNA and antisense RNA are antiparallel.

Thus, by “symmetrical nucleotide ordering” is meant that chemically the same natural nucleotides (not possessing this symmetry in their chemistry) form the palindrome half. These true DNA palindromes are similar to linguistic ones. The following example can clarify this point—gene and mirror nucleotide ordering in artificial DNA are shown (SEQ ID NOS 6-9 are disclosed respectively in order of appearance):

| | | |
|---|---|---|
| gene | axis | mirror-inverted artificial DNA |

| | | | | |
|---|---|---|---|---|
| (1) | 5′ . . . CTGATAATGTATC . . . 3′ | \| | (3) | 5′ . . . CTATGTAATAGTC . . . 3′ |
| (2) | 3′ . . . GACTATTACATAG . . . 5′ | \| | (4) | 3′ . . . GATACATTATCAG . . . 5′. |

pcRNA in this example is complementary to the selected region of mRNA in the same polarity (SEQ ID NOS 10 & 11 respectively in order of appearance):

| | |
|---|---|
| (1) | 5′ . . . CUGAUAAUGUAUC . . . 3′ (mRNA) |
| (4) | 5′ . . . GACUAUUACAUAG . . . 3′ (pcRNA).” |

Such DNA texts do not happen in nature, but they do possess biological activity on the RNA level.

Integration refers to the integration of the genetic construct in the proper position and orientation with respect to the different types of regulatory elements in a chromosomal context, in order to obtain better expression all or particular stages of development.

### EXAMPLE 1

Generation of Kruppel (Kr) Phenocopies via Injections of In Vitro Synthesized pcRNA into Drosophila Embryos Complementary in the Same 5′-3′ Polarity to the mRNA.

Kr is a homeotic gene, that is active in zygote and controls segment formation at the early embryonic stage of Drosophila development. Kr was selected as a model, allowing one to observe the early development in a multicellular organism. Kr mutants contains deletions of the adjacent thoracic and anterior abdominal segments. Phenotypically, this is observed just after cuticula formation and hatching of the larvae. Kr mutants have deletions of the adjacent thoracic segments as well as deletions of from one to several anterior abdominal segments. Sometimes, deletions are present in the development of actopic tracheal ending in the anterior part of the larva [E. Weischaus, C. Nusslein-Volhard, H. Kluding, Development, 1984, vol. 104, p. 172]. Thereafter, the Kr mutants have a unique phenotype developed as early as the first day, which provides an advantage for study of the effect of RNA on a phenotype. An additional reason for the use of this model was that the study by antisense RNA injections already performed [U. B. Rosenberg, A. Preiss, E. Seifert, H. Jackle, D. C. Knippe, Nature, 1985, vol. 313, p. 703].

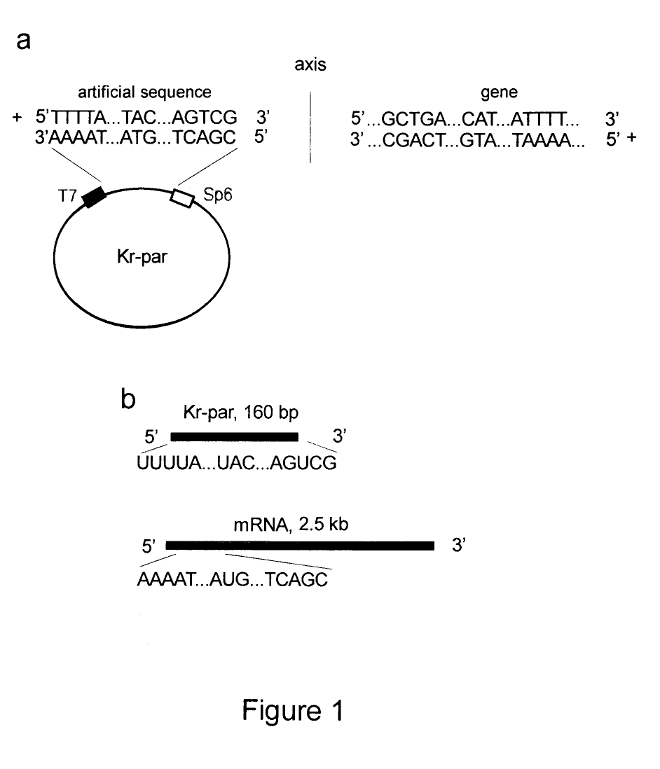

An artificial DNA 160-bp sequence encoding pcRNA possessing the mirror nucleotide ordering in respect to the region of the Kr gene was chemically synthesized (FIGS. 1 a, b). It should be stressed that antisense RNAs are synthesized on the non-coding strand of the same gene, while pcRNA may be synthesized only on the heterologous artificial DNA possessing the mirror order of nucleotide sequence.

The chemically synthesized artificial DNA was inserted into the pGEM-1 vector in the orientation allowing pcRNA synthesis in vitro using T7 RNA polymerase. pcRNA was termed Kr-par, because it is complementary in a parallel orientation to Kr mRNA.

Embryos of the Oregon RC line were injected with pcRNA samples in the posterior pole at the syncytial stage and incubated under the water at 25° C. for 18-24 hours. Then, cuticula mounts were prepared and studied under the phase-contrast microscope.

Development of the larvae possessing the typical Kr phenotype were observed. In control experiments, the development of normal larvae were observed following injections of RNA synthesized on the opposite strand of the same construct with SP6 RNA polymerase.

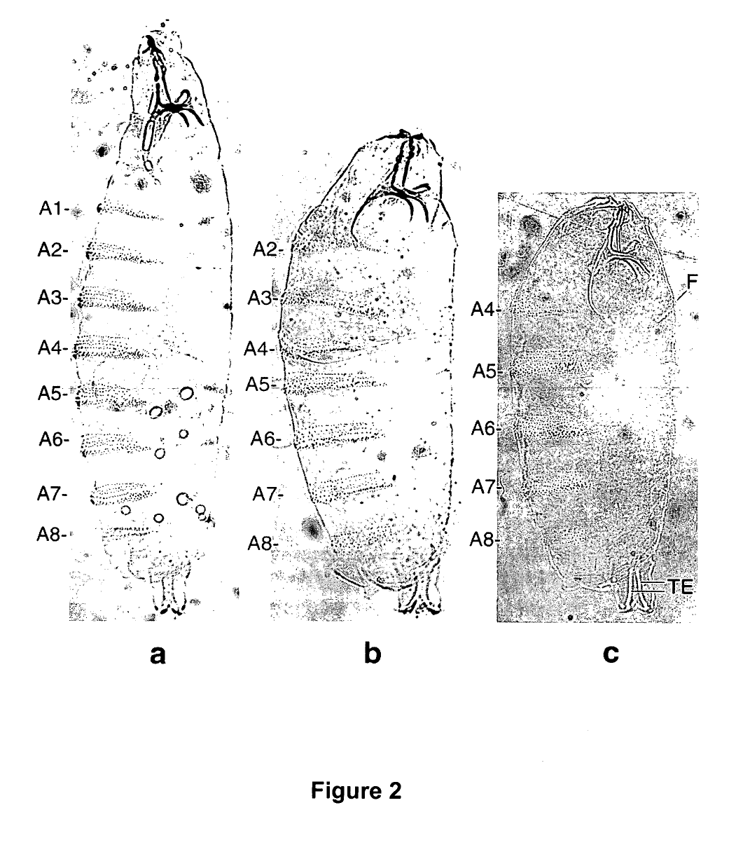

FIG. 2 shows the normal larva (a) and two larvae developed after injections of Kr-par preparation (b, c). The latter have deletions of the thoracic and one or three abdominal segments as well as of the actopic tracheal ending in the anterior part of the larva (typical for Kr phenotype). The frequencies of observed phenocopies are about the same value as after injections with asRNA [U. B. Rosenberg, A. Preiss, E. Seifert, H. Jackle, D. C. Knippe, Nature, 1985, vol. 313, p. 703]. This demonstrates that pcRNA affects the expression of a key gene for differentiation in a multicellular organism, and a directed change of genetical properties of organism is achieved.

Thus, short stretches of DNA possessing mirror inversions of nucleotide sequences in respect to selected genes do possess biological activity, as their transcripts (pcRNAs) are capable of inducing a strong gene-specific silencing. In this way, they are able to change the genetic properties of an organism.

### EXAMPLE 2

Gene-Specific Silencing in Human Cells Induced by In Vivo Expression of the RNA Stretch Complementary in the Same 5′-3′ Polarity to the mRNA of the Renilla Luciferase Gene.

The model system used included human cultured HEK 293, psiCHECK-2 vector (Promega), containing two luciferase genes (from fire fly and from Renilla) and specified 30 nt long pcRNA corresponding to the Renilla luciferase mRNA. It is known that HEK 293 cells are generated by transformation of human embryonic kidney cell cultures (hence HEK) with sheared adenovirus 5 DNA, and were first described in 1977 [F. L. Graham J. Smiley, W. C. Russell, R. Nairn, J. Gen. Virol., 1977, vol. 36, p. 59]. It was later described that HEK 293 cells may have originated from a rare neuronal cell in kidney cell cultures.

The Promega psiCHECK-2 vector (AC number AY535007) was originally designed to provide a quantitative and rapid method for gene-specific silencing by RNA interference. Renilla luciferase gene is used as a primary reporter gene. The firefly gene was used in the present experiments as an internal control for normalization of transfection data. The vector allowed for the monitoring of changes of expression of the reporter Renilla luciferase gene if a target gene segment was fused to the Renilla reporter gene in the multiple cloning region located downstream of the Renilla coding region. It is assumed that as result of recognition of a target gene region by siRNA, the RNA interference towards a target gene leading to the the cleavage and subsequent degradation of fusion mRNA occurs. This was measured by decreased Renilla luciferase activity.

FIGS. 3a-3d, show the experimental design used. The Renilla gene has cloning sites located between the coding region and synthetic polyadenylation stretch. These cloning sites were designed by Promega for insertion of different DNA fragments (target gene regions) for testing the efficiency of siRNAs. Cloning sites XhoI and NotI were used for insertion of 30 bp sequences specifying within Renilla luciferase mRNA the different RNA stretches containing bases complementary to the same mRNA region either in parallel or in antiparallel orientation. FIG. 3a shows the region of mRNA, as well as both stretches encoded by the insertions. The 30 nt long RNA segment that is complementary in parallel was designated as “pcRNA” and the segment that is complementary in antiparallel orientation was designated as “asRNA”.

To prepare the constructs, the chemically synthesized overlapping pairs of oligonucleotides were used:

for the construct expressing pcRNA-5′cccctcgagCTCGGGGTCGTACACCTTGGAAG 3′ (SEQ ID NO: 12) and 5′ cccgcggccgcACCATGGCTTCCAAGG 3′ (SEQ ID NO: 13); for the construct expressing asRNA-5′ cccctcgagTGGTACCGAAGGTTCCACATG 3′ (SEQ ID NO: 14) and 5′ cccgcggccgcGAGCCCCAGCATGTGGAACC 3′ (SEQ ID NO: 15) (artificial sites for XhoI or NotI endonucleases are shown by lowercase). Both pairs of oligonucleotides correspond to the 691-720 bp region of Renilla gene (numberings as indicated in AY535007). About 1 μg of nucleotides of each pair were mixed in 20 μl of the solution containing 50 mM NaCl, 40 mM tris-HCl, pH 7.4, 10 mM MgCl₂, 10 mM β-mercaptoethanol, and then cooled over 10 minutes from 65° C. to 20° C., to anneal the overlapping regions. Then the filling in of the 5′-protruding ends was performed after 40 μM and 1 unit of Klenow Fragment of DNA polymerase I was added to the mixtures of each dNTP. After incubation at room temperature for 30 minutes, the enzyme was inactivated by heating at 65° C. for 10 minutes. Then 2 μl each of 0.1M EDTA and 1M NaCl were added to the mixtures, as well as 1 μl of glycogen (1 mg/ml). The DNA was precipitated by the addition of 3 vol. of ethanol followed by incubation in an ice bath and shaking for 7 minutes. After centrifugation in an Eppendorf centrifuge at 12000 rpm for 10 minutes, the ethanol was discarded and precipitate dried for 5 minutes. Then the DNA precipitate was dissolved in the buffer containing 50 mM NaCl, 40 mM tris-HCl, pH 7.4, 10 mM MgCl₂, 10 mM β-mercaptoethanol and 1 units of each NotI and XhoI restriction enzymes. After incubation at 37° C. for 1 hour the enzymes were heat-inactivated and DNA was ethanol precipitated as described above. Finally about 10 ng of final DNA was dissolved in the 20 μl of the solution containing 100 mM NaCl, 40 mM tris-HCl, pH 7.4, 10 mM MgCl₂, 10 mM β-mercaptoethanol, 7 μM ATP. Then 60 ng of psiCHECK-2 vector digested by NotI and XhoI enzymes and 1 unit of T4-DNA ligase were added. After incubation at room temperature for 1-16 hours the mixture was used for the standard procedures of transformation of JM103 E. coli cells and selection of recombinants by the colony hybridization. The clones obtained were sequenced using the specific primer 5′ GAGAAAATTAGTAGATTTC 3′ (SEQ ID NO: 16).

Schematically, the insertions inside the psiCHECK-2 vector are shown on FIGS. 3b and 3C. The clones obtained were designated as “par” and “as”. The clones express long RNA molecules (more than 1200 nt long), having complementary regions coming from the insertions and corresponding to Renilla luciferase mRNA fragment from 691 to 720 nt (numberings as indicated in the psiCHECK-2 vector) at their 3′ ends. It was expected that the complementary regions could form short duplexes. The geometry of such molecules is shown at FIG. 3d. These abnormal double-stranded regions in Renilla mRNA molecules may be used by very sensitive cellular mechanisms of monitoring of RNA molecules. As a result, the mechanisms of silencing could then be used. It is well known that 30 bp long antiparallel RNA-RNA duplexes (dsRNAs) are very strong triggers of RNAi mechanisms (up to 100 times stronger than 22 bp sequences) [Dong-Ho Kim, Mark A Behlke, Scott D. Rose, Mi-Sook Chang, Sangdun Choi and John J. Rossi, Nature Biotechnology, 2004, vol. 23, p. 222]. Thus, the construct as expressing the regular dsRNA was used as a positive control in experiments with the construct par.

The construct for transfection of human HEK293 cells was used. The cells were seeded into 96-well plate at a density 4000-9000 cells/well one day prior to the experiment. Par and as DNAs, as well as DNA of the original psiCHECK-2 vector, were used for preparation of lyposomes. 2-4 ng of individual DNA in 35 μl of serum-free medium 0.3 μl of TransFast transfection reagent (Promega) was added per well and incubation was performed for 15 minutes at room temperature. The medium was removed from above the seeded cells and the suspension of liposomes was added to the cells. The incubation was performed at 37° C. for 1 hour. Then, 80 μl of serum-containing medium was added in each well and incubation from 24 to 96 hours was performed. Firefly and Renilla luciferase activities were measured using the Dual-Luciferase Reporter Assay System (Promega) and the Reporter Microplate Luminometer (Turner BioSystems). The Renilla luciferase data have been normalized to fire fly luciferase data. Excel and Origin software was used for data analysis and graphing. A series of experiments studying the expression of Renilla using the constructs was performed.

The inhibition of Renilla luciferase by par and as constructs (FIG. 4) was observed. In all of the experiments (all not shown) the inhibition by par construct was higher than the inhibition by as. After a longer incubation of the cells, a stronger inhibition was observed (FIG. 5). Thus, some mechanisms of gene-specific silencing are developed during longer incubation. From this data, the conclusion was drawn that pcRNA is a powerful tool for gene-specific silencing in human cells.

Two further genetic constructs designed similarly as shown in FIG. 3, but possessing sequences targeting regions 1571-1600 or 911-940 of the Renilla mRNA were tested by the same way. Again the par-2 or par-3 constructs demonstrated stronger inhibition than the corresponding as-2 or as-3 constructs.

### EXAMPLE 3

RNA Interference in Cells of Escherichia coli with the Help of RNA Molecules Complimentary in Parallel Direction to the mRNA of the Lon Gene, Synthesized In Vivo.

The lon gene was selected as a model, because it is one of key factors in the regulation of many processes in E. coli cells.

An artificial 95 b-long DNA sequence (bp denotes a base pair in the DNA or RNA) having a mirror-symmetrical nucleotide sequence with respect to the corresponding region of the lon gene was chemically synthesized.

This DNA was used for producing a construct based on a pUC12 vector in which the chain expressing the pcRNA parlon is under control of the lac promoter (FIGS. 1a and 1b).

The resulting construct was introduced into E. coli cells by transformation.

The impact of the pcRNA parlon on the activity of the endogenous lon gene was monitored by the effect of the latter on the operation of the lux-reulon introduced into the E. coli cells from Vibrio fischeri cells. Lon protease is a negative regulator of the lux-regulon, because it specifically degrades the LuxR protein. The latter in a complex with an autoinductor triggers the synthesis of proteins responsible for luminescence. Pr and pl promoters in the lux-regulon operate on different chains, the genes are shown by light rectangles (FIG. 1c). The actively operating lon gene brings about repression of the lux-region transcription, and this manifests itself phenotypically as inhibition of the luminescence. On the contrary, silencing of the ion gene leads to an increase of he LuxR concentration and, consequently, to activation of the lux-regulon transcription, this resulting in a considerable enhancement of the luminescence of the cells. Lux-regulon, as 16 kb BamHI DNA fragment from Vibrio fischeri was introduced into lon⁺ cells of E. coli K12 AB1157 and also into lon⁻ cells of E. coli K12 AB1899 (lon1).

Into the resulting lon lon⁺ cells a construct is also introduced separately, which is produced on the basis of the pUC12 vector and is capable of expressing the pcRNA parlon or the initial pUC12 plasmid containing no insert. Silencing of the lon gene is determined from the enhancement of the luminescence of the cells. The luminescence intensity of the cells expressing the pcRNA parlon increases by several orders of magnitude compared with control containing the initial pUC12 plasmid (FIG. 2).

In growing parlon transformants on solid media, the development of “mucous” colonies was observed, this being typical of the lon⁻ phenotype or silencing of the lon gene.

While the present invention has been described with reference to specific embodiments, this application is intended to cover those various changes and substitutions that may be made by those of ordinary skill in the art without departing from the spirit and scope of the appended claims.

The publications discussed herein are provided solely for their disclosure prior to the filing date of the present application. Nothing herein is to be construed as an admission that the present invention is not entitled to antedate such publication by virtue of prior invention. Further, the date of publication may be different from the actual publication dates, which may need to be independently confirmed.

1. A method for changing the genetic properties of an eukaryotic organism by altering or preventing expression of a target gene, comprising:
(a) selecting a target gene, and
(b) introducing into the organism pcRNA molecules that are complementary in a parallel orientation to mRNA of the target gene or a fragment thereof.

2. The method of claim 1, wherein the pcRNA molecules that are complementary in a parallel orientation to mRNA of the target gene or fragment thereof are synthesized in vitro prior to introduction into the organism.

3. The method of claim 2, wherein the pcRNA molecules that are complementary in a parallel orientation to mRNA of the target gene or fragment thereof are synthesized using an artificial DNA sequence as a template, wherein the artificial DNA sequence encodes pcRNA.

4. The method of claim 2, wherein the pcRNA molecules that are complementary in a parallel orientation to mRNA of the target gene or fragment thereof are introduced into the organism via injection.

5. The method of claim 1, wherein the pcRNA molecules that are complementary in a parallel orientation to mRNA of the target gene or fragment thereof are introduced into the organism via introduction into the organism of a DNA construct that expresses the pcRNA molecules that are complementary in a parallel orientation to mRNA of the target gene or fragment thereof in vivo.

6. The method of claim 5, wherein the DNA construct comprises a suitable promoter and a DNA sequence encoding pcRNA,
wherein said DNA sequence possesses symmetrical nucleotide ordering with respect to the nucleotide sequence of the target gene; and
wherein said DNA sequence is under control of the promoter.

7. The method of claim 6, wherein said DNA construct further comprises sequences that enable integration of the DNA construct into the chromosome of the organism and the proper expression of the DNA construct.

8. The method of claim 1, wherein the pcRNA molecules that are complementary in a parallel orientation to mRNA of the target gene or fragment thereof are introduced into the organism via ex vivo gene therapy.

9. The method of claim 8, wherein said ex vivo gene therapy comprises:
(a) removing cells from the organism,
(b) transforming the cells with a vector comprising a DNA construct that expresses the pcRNA molecules that are complementary in a parallel orientation to mRNA of the target gene or fragment thereof in vivo, to produce genetically modified cells, and
(c) injecting the genetically modified cells into the organism.

10. The method of claim 9, wherein the DNA construct comprises a suitable promoter and a DNA sequence encoding pcRNA, wherein said DNA sequence encoding pcRNA is under control of the promoter.

11. The method of claim 10, wherein the DNA construct further comprises sequences that enable integration of the DNA construct into the chromosome of the organism and the proper expression of the DNA construct.

12. The method of claim 1, wherein said pcRNA molecules that are complementary in a parallel orientation to mRNA of the target gene or a fragment thereof interacts with the mRNA of the target gene, thereby altering or preventing expression of the target gene.

13. The method of claim 1, wherein the target gene has undesirable activity.
